(19) United States
(12) Patent Application Publication
(10) Pub. No.: US 2025/0204808 A1
(43) Pub. Date: Jun. 26, 2025

(54) A METHOD AND A SYSTEM FOR MONITORING A TREATMENT AREA OF A PATIENT

(71) Applicant: 360MEDLINK INC., Montréal (CA)

(72) Inventors: Jean-Manassé THEAGÈNE, Montréal (CA); Ahmed TAHA AROUA, Saint-Hyacinthe (CA)

(21) Appl. No.: 18/848,300

(22) PCT Filed: Mar. 16, 2023
(86) PCT No.: PCT/CA2023/050335

§ 371 Date: Sep. 18, 2024

Related U.S. Application Data
(60) Provisional application No. 63/322,071, filed on Mar. 21, 2022.

(51) Int. Cl. A61B 5/107; A61B 5/00; G06T 7/62

(52) U.S. Cl. CPC A61B 5/107; A61B 5/0059; A61B 5/4848; G06T 7/62; G06T 2207/10004; G06T 2207/30088

(57) ABSTRACT

When treating a patient suffering from peripheral neuropathic pain by cutaneous administration of, for example, capsaicin, monitoring involves marking the treatment area on a skin of the patient and/or on a substrate which is separate from the skin of the patient, acquiring pictures of the treatment area and a reference symbol, detecting closed contour areas and the reference symbol, determining a closed contour area size, grouping the closed contour areas according to their shape, determining a characteristic area size for each group of contour areas, and selecting a group of closed contour areas, and storing the shape and the characteristic area size of the selected group in a database.

### CROSS-REFERENCE TO RELATED APPLICATIONS

This application claims priority from U.S. provisional patent application 63/322,071 filed Mar. 21, 2022, which is hereby incorporated by reference.

### TECHNICAL FIELD

The present invention relates to the field of monitoring a treatment area of a patient which patient is treated by cutaneous administration of a substance in the treatment area. Monitoring the treatment area may comprise performing measurements of a treatment area and storing data derived from such measurements.

### BACKGROUND

The invention may find application in monitoring a treatment area of a patient suffering from skin irritations, skin infections—in particular bacterial skin infections or fungal skin infections (mycosis)—skin ulcers, burns, eczema, psoriasis or pruritus.

The invention finds particular application in monitoring a treatment area of a patient suffering from peripheral neuropathic pain which patient is treated by cutaneous administration of capsaicin in the treatment area. Capsaicin {i.e. (E)-8-methyl-N-vanillyl-6-nonenamide, trans-8-methyl-N-vanillyl-6-nonenamide, 6-nonenamide, (E)-N-[(4-hydroxy-3-methoxyphenyl)methyl]-8-methyl} occurs naturally in chili peppers and is a highly selective agonist for the TRPV1 (Transient Receptor Potential Vanilloid 1) receptor, which is considered one of the main receptors in the transmission and modulation of pain signals. Capsaicin is typically administered in the form of a transdermal patch (Qutenza®) containing a very high dose (8%) which leads to overstimulation of the TRPV1 receptors. Before application, typically, a healthcare professional cuts the size of the patch to the size of the painful area to be treated. The patch, usually, remains on the treatment area of the skin for 30 to 60 minutes.

After a single application, the patch provides effective pain relief for three months. However, a typical therapy includes subsequent applications of patches. Experience has shown that treatment area shape and/or size may vary with each further application. Preferably, the treatment area size decreases with further applications.

In light of the above, it would be advantageous to improve monitoring of a cutaneous therapy of a patient in particular suffering from peripheral neuropathic pain.

### SUMMARY

To better address this concern, in a first aspect of the invention a method is presented for monitoring a treatment area of a patient, in particular a patient suffering from peripheral neuropathic pain, which patient is treated by cutaneous administration of a substance, in particular comprising capsaicin, in the treatment area, the method comprising the following method steps:
in a marking step, the treatment area is marked on a skin of the patient and/or on a substrate which is separate from the skin of the patient;
in an acquisition step, two or more pictures of a scene are acquired, wherein the scene comprises the treatment area marked in the marking step and a predetermined reference symbol having a predetermined reference area size;
in a detection step, closed contour areas and the predetermined reference symbol are detected in each of the pictures acquired in the acquisition step;
in a determination step, a closed contour area size is determined for each closed contour area detected in the detection step from the respective detected closed contour area and the predetermined reference symbol detected in the same picture as the closed contour area;
in a grouping step, the closed contour areas detected in the detection step are grouped according to their shape, thereby providing one or more groups of closed contour areas;
in a characterization step, a characteristic area size is determined for each group of contour areas identified in the grouping step from the closed contour area sizes assigned to the closed contour areas of each group;
in a selection step, a group of closed contour areas is selected;
in storing step, the shape and the characteristic area size of the selected group is stored in a database.

According to another aspect of the invention a system is presented for monitoring a treatment area of a patient, in particular a patient suffering from peripheral neuropathic pain, which patient is treated by cutaneous administration of a substance, in particular comprising capsaicin, in the treatment area, wherein the treatment area is marked on a skin of the patient and/or on a substrate which is separate from the skin of the patient,
the system comprising an acquisition device configured to acquire two or more pictures of a scene, wherein the scene comprises the marked treatment area and a predetermined reference symbol having a predetermined reference area size;
the system comprising a processing device configured to carry out the following method steps:
   in a detection step, closed contour areas and the predetermined reference symbol are detected in each of the pictures acquired by the acquisition device;
   in a determination step, a closed contour area size is determined for each closed contour area detected in the detection step from the respective detected closed contour area and the predetermined reference symbol detected in the same picture as the closed contour area;
   in a grouping step, the closed contour areas detected in the detection step are grouped according to their shape, thereby providing one or more groups of closed contour areas;
   in a characterization step, a characteristic area size is determined for each group of contour areas identified in the grouping step from the closed contour area sizes assigned to the closed contour areas of each group;
   in storing step, the shape and the characteristic area size of a selected group is stored in a database.

Both the inventive method and system provide improved monitoring of a cutaneous therapy of a patient in particular suffering from peripheral neuropathic pain. The treatment area, which is typically the area of the skin the patient is suffering pain at, is marked directly on the skin of the patient and/or on a separate substrate. Marking the treatment area facilitates providing the substance, in particular capsaicin, exactly to the treatment area without adversely affecting the skin outside the painful area. If a transdermal patch is used to administer the substance the patch may be cut in the form of the treatment area. Marking the treatment area each time the patient is treated also serves monitoring long-term development of the painful area. The invention includes acquisition of multiple pictures of a scene comprising both the marked treatment area and a predetermined reference symbol. Taking multiple pictures of the same scene enables more reliable detection of the treatment area. In fact, treatment area shape and size can be derived from more than one picture. The predetermined reference symbol has a predetermined reference area size. In each of the acquired pictures closed contour areas and the reference symbol are detected. Then, the closed area sizes of the detected closed areas contours are determined with reference to the reference symbol, in particular the predetermined reference area size of the reference symbol. The detected closed contour areas are also grouped according to their shape, thereby providing one or more groups of closed contour areas. The grouping step may ideally result in just one group. However, due to possible inaccuracies during acquisition and/or detection of the closed contour areas, artefacts may be generated. Those artefacts may result in one or more additional groups of closed contour areas. By grouping the closed contour areas selecting the correct closed contour relating to the treatment area is facilitated. For each of the determined groups a characteristic area size is determined. From the determined groups of closed contour areas one group is selected, in particular the group that reflects the treatment area best. The selection can be done by a user, e.g., a healthcare professional. Then, the shape and the characteristic area size of the selected group is stored in a database.

According to the invention, the grouping step may be carried out before the determination step or the determination step may be carried out before the grouping step. Alternatively, the grouping step and the determination step may be carried out simultaneously.

Preferred embodiments of the invention are subject of the dependent claims and will be discussed in the following.

According to a preferred embodiment of the invention, the substrate is a foil or a tracing paper or a patch, in particular a patch comprising the substance, preferably capsaicin. The substrate is preferably placed on the patient's skin during the marking step. Preferably, the substrate is provided as a transparent substrate so that the treatment area may be marked on the substrate while the patient's skin is visible through the substrate. For example, the treatment area may be pre-marked directly on the patient's skin, the transparent substrate may be placed on the skin and the markings may be transferred to the substrate using a marking means, e.g., a pen, thereby creating a marked treatment area on the substrate. Marking the treatment area on a substrate which is separate from the skin comes with the advantage, that the substrate may be removed from the skin and placed flat on a flat surface during acquisition of the pictures. Thereby, curvatures of the substrate may be eliminated and determination of the characteristic area size may be facilitated. Alternatively, the treatment area may be marked directly on the skin of the patient.

According to a preferred embodiment of the invention, in the marking step, the treatment area is marked as an outline or is marked as a colored area. Marking can be done by a user, e.g., a healthcare professional, using a marking means, e.g., a pen.

According to a preferred embodiment of the invention, the predetermined reference symbol is provided on the skin or on the substrate or as a separate element. When provided on the skin, the reference symbol may be implemented as a temporary tattoo or a sticker. When provided on the substrate, the reference symbol may be printed on the substrate or embedded into the substrate. Preferably, the reference symbol is provided as a separate element, e.g., as a card or sheet. Regardless of the way the reference symbol is provided, it may comprise a machine-readable code, in particular a one-dimensional code, e.g., a bar code, or a two-dimensional code, e.g., a QR-code. Alternatively, the reference symbol may be the machine-readable code.

According to a preferred embodiment of the invention, in the acquisition step, an orientation of an acquisition device acquiring the pictures is determined, in particular by a motion sensor. The motion sensor may be part of the system, in particular of the acquisition device. Based on the orientation determined the treatment area size may be corrected to account for non-parallelism of the treatment area and an image area of the acquisition device. Alternatively, or additionally, a warning may be generated if the orientation of the acquisition device deviates more than a predetermined threshold from the orientation of the treatment area and/or an orientation of a flat surface the substrate is placed on during acquisition of the pictures. To assist the user of the acquisition device, the acquisition device may display an indication of the determined orientation and/or an indication if the determined orientation is within a predefined range. The range may be predefined by the user prior to carrying out the acquisition step so that each user may create and use his own range settings.

According to a preferred embodiment of the invention, in the grouping step, the closed contour areas detected in the detection step are compared to each other. Preferably, the shape and/or the closed contour area size of the closed contour areas detected in the detection step, are compared to each other. Comparing the closed contour areas may include comparing two detected closed contour areas and determining a degree of similarity between the two closed contour areas. Pairs of closed contour areas having a high degree of similarity are likely to belong to the same group.

According to a preferred embodiment of the invention, in the grouping step, the closed contour areas detected in the detection step are grouped according to their shape into groups having similar shape. By grouping the closed contour areas into groups of similar shape it is possible to remove closed contour areas resulting from artefacts very efficiently. This is due to the fact that closed contour areas resulting from artefacts typically have shape that distinguish from the shape of the treatment area.

According to a preferred embodiment of the invention, the predetermined reference symbol is used as a calibration standard for determining the closed contour area size in the respective picture. The predetermined reference symbol has a predetermined reference area size. Therefore, the closed contour areas detected in each of the acquired pictures can be calibrated using the predetermined reference area size. Thereby, comparability between different pictures of the same scene is increased.

According to a preferred embodiment of the invention, the characteristic area size is a median area size. When using the median area size as characteristic area size, the effect of very large or very low area sizes on the characteristic area size can be reduced. Preferably, for each group of contour areas extreme area size values are removed prior to determination of the characteristic area size. For example, are size values exceeding a predetermined maximum area size and/or area size values below a predetermined minimum area size are removed prior to determination of the characteristic area size. Alternatively, the characteristic area size is a mean area size.

According to a preferred embodiment of the invention, that the selection step comprises displaying the one or more groups determined in the grouping step to a user, preferably a health care professional. The one or more groups may be displayed in a display device. The user may easily assess the groups and may select the one that matches the treatment area best.

According to a preferred embodiment of the invention, that the acquisition step and the detection step and the determination step and the grouping step and the characterization step are repeated before a group of contour areas is selected in the selection step. Repeating the before mentioned steps may be started upon request by the user. For example, a user which considers none of the groups identified in the grouping step to represent the treatment area may initialize another procedure to acquire and determine the treatment area.

According to a preferred embodiment of the invention, that a photo of the skin of the patient is taken, wherein the photo is stored in the database in the storing step. The photo of the skin of the patient can be taken prior to or after the acquisition of the pictures in the acquisition step. The photo is preferably assigned to the dataset including the shape and the characteristic area size of the treatment area. When displaying the dataset, the photo can facilitate understanding the position of the treatment area on the patient's skin.

According to a preferred embodiment of the invention, that a pain intensity information is received, wherein the pain intensity information is stored in the database in the storing step. The pain intensity information may be provided as a score on a scale. The pain intensity information is preferably assigned to the dataset including the shape and the characteristic area size of the treatment area. When displaying the dataset, the pain intensity information can facilitate understanding the degree of pain observed by the patient before treatment with the substance, in particular capsaicin.

According to a preferred embodiment of the invention, the method further includes a calculation step, in which a difference between the characteristic area size of the selected group and a predetermined characteristic area size is calculated. Thereby, a deviation from the predetermined characteristic area size may be indicated to the user. The predetermined characteristic area size may be a previously determined and stored characteristic area size, for example a characteristic area size determined at a previous treatment situation, e.g., a treatment situation one or more month ago. In a typical scenario, the characteristic area size decreases with ongoing treatments. Thus, the calculation step may serve to show the effect of recurrent treatments with the substance, in particular capsaicin.

It will be appreciated that the monitoring method can be used to guide or influence a subsequent application or cutaneous administration of capsaicin (or the like). In some embodiments, there is provided a method for treatment of a patient suffering from peripheral neuropathic pain. The method may comprise cutaneous administration of capsaicin in a treatment area, monitoring said treatment area of said patient, determining a size and a location of a subsequent treatment area based on characterization of closed contour areas from the monitoring and cutaneous administration of capsaicin in the subsequent treatment area. The monitoring can involve at least, in an acquisition step, two or more pictures of a scene are acquired, wherein the scene comprises the treatment area, and, in a detection step, detecting the closed contour areas.

### BRIEF DESCRIPTION OF THE DRAWINGS

These and other aspects of the invention will be apparent from and elucidated with reference to the embodiments described hereinafter.

FIG. 1 is a schematic representation of a method in accordance with a first embodiment of the invention.

FIG. 2 is a picture of a scene acquired as part of the method according to FIG. 1;

FIG. 3 is a series of pictures acquired as part of the method according to FIG. 1;

FIG. 4 is a set of groups determined as part of the method according to FIG. 1; and

FIG. 5 is a information stored in the database according to the method of FIG. 1.

### DETAILED DESCRIPTION

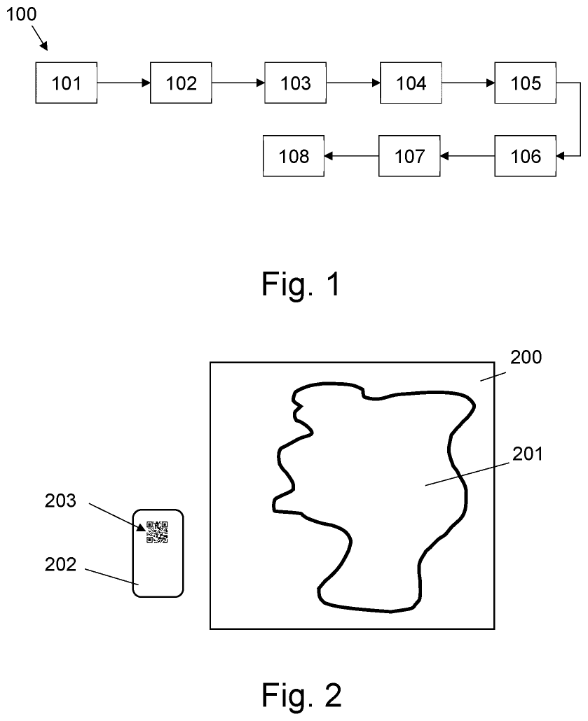

FIG. 1 illustrates an embodiment of the invention in the form of a method 100 for monitoring a treatment area of a patient suffering from peripheral neuropathic pain which patient is treated by cutaneous administration of a substance, in particular comprising capsaicin, in the treatment area. The method improves monitoring of the cutaneous therapy of the patient and involves several steps that will be described in the following with reference to FIG. 1. Although, the method is described with reference to administration of capsaicin to a patient suffering from peripheral neuropathic pain, the method may find application in monitoring treatment of patients suffering from skin irritations, skin infections—in particular bacterial skin infections or fungal skin infections (mycosis)—skin ulcers, burns, eczema, psoriasis or pruritus. In such applications a substance other than capsaicin may be used in the treatment area.

First, in a marking step 101, the treatment area is marked on a skin of the patient or on a substrate which is separate from the skin of the patient. Preferably, in a first sub-step of the marking step 101, a painful area is marked on the patient's skin, e.g., using a pen. Then, in a second sub-step of the marking step 101, a transparent substrate is placed on the patient's skin in the region of the painful area. The markings on the skin are then traced with a pen marking the treatment area 201 as an outline on the substrate 200, see FIG. 2. The substrate 200 is preferably provided as a patch that comprises an active substance, in particular capsaicin.

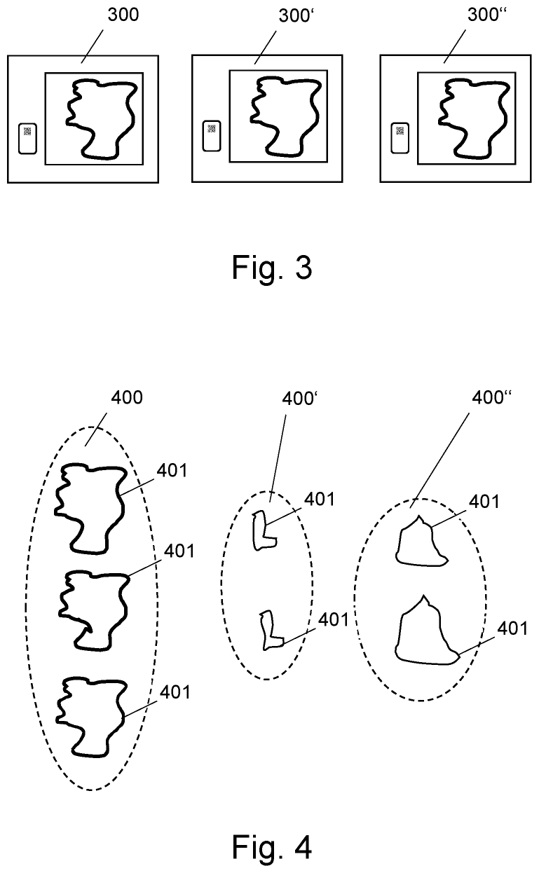

In an acquisition step 102 following the marking step 101, the substrate 200 is placed flat on a flat surface. Further, a predetermined reference symbol 202 having a predetermined reference area size is placed next to the substrate or on the substrate so that pictures of both, the treatment area 201 marked on the substrate 200 and the predetermined reference symbol 202 can be acquired as a part of a common scene. According to the embodiment shown in FIG. 2, the predetermined reference symbol 202 is provided as a separate element in the form of a card. Here, the predetermined reference symbol 202 comprises a code 203, in particular a two-dimensional code. The code 203 is machine readable, in particular optically machine readable. The reference symbol 202, in particular the code 203 of the reference symbol 202 is used as a calibration standard for determining the closed contour area size in the respective picture in a later step of the method according to the invention. The acquisition step 102 includes that at least two, preferably more than two pictures of the scene are acquired. The pictures 300 may be acquired as part of a series of pictures 300 triggered by the same event, e.g., a user of the system pushing a button. An exemplary set of pictures 300, 300′, 300″—here three pictures 300, 300′, 300″—is depicted in FIG. 3. Acquisition is preferably carried out using an acquisition device, in particular a camera, preferably a digital camera. Optionally, during acquisition, an orientation of the acquisition device may be determined, in particular by a motion sensor. The acquisition may indicate to the user if the acquisition device is oriented in parallel to the substrate or with an angular offset to the substrate.

In detection step 103, subsequent to the acquisition step 102, closed contour areas and the predetermined reference symbol 202, in particular the code 203 of the reference symbol 202, are detected in each of the pictures acquired in the acquisition step. As a result, the number of closed contour areas is at least equal the number of pictures acquired. However, typically the number of closed contour areas exceeds the number of pictures acquired due to artefacts on the acquisition step or the detection step. For example, if three pictures have been acquired in the acquisition step, the number of closed contour areas may be seven.

In a determination step 104, subsequent to the detection step 103, a closed contour area size is determined for each closed contour area detected in the detection step from the respective detected closed contour area and the predetermined reference symbol detected in the same picture as the closed contour area. Here, the reference symbol 202, in particular the code 203 of the reference symbol 202, is used as a calibration standard for determining the closed contour area size. This is because, the symbol 202, in particular the code 203 of the reference symbol 202, has a predetermined reference are size. The closed area sizes in each of the pictures can be calculated in relation to the reference area size of the reference symbol in the respective picture.

In a grouping step 105, the closed contour areas detected in the detection step are grouped according to their shape, thereby providing one or more groups of closed contour areas. As a result, different groups of closed contour areas having similar shape are received. An exemplary set of closed contour areas 401 that have been grouped into three groups 400, 400′, 400″ is depicted in FIG. 4. As visible from FIG. 4 the closed contour areas 401 of each group 400, 400′, 400″ have similar shape.

According to a variant of the embodiment depicted in FIG. 1, the grouping step 105 may be carried out before the determination step 104. According to yet another variant, the determination step 104 and the grouping step 105 may be carried out simultaneously.

In a characterization step 106 following the grouping step 105, a characteristic area size is determined for each group of contour areas identified in the grouping step 105. Determination is carried out based on the closed contour area sizes assigned to the closed contour areas of each group. According to the embodiment, the characteristic area size is a median area size, in other words, the middle value of all closed contour area sizes of a group (not the mean area size).

The characterization step 106 is followed by a selection step 107 in which a group of closed contour areas is selected, e.g., by the user. As part of the selection step 107, the characteristic area size together with a symbolic shape of the group may be displayed to the user. The user may select the group by choosing the respective symbolic shape.

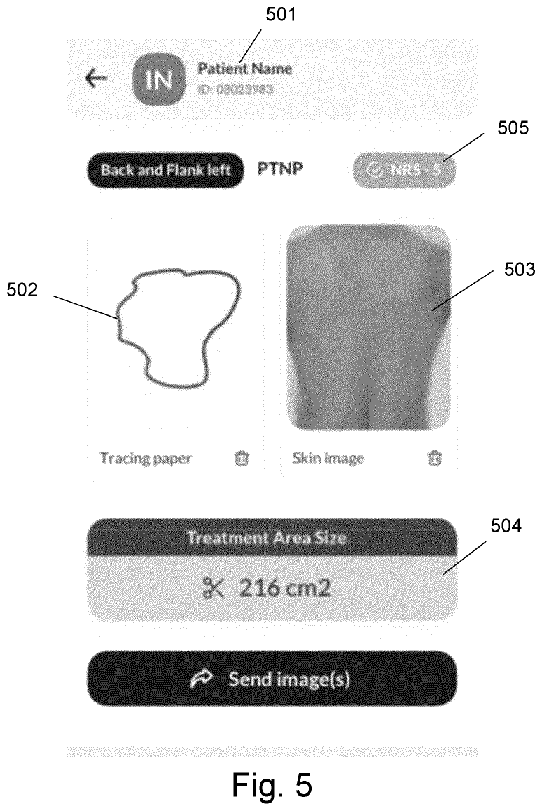

After selection, in a storing step 108, the shape and the characteristic area size of the selected group is stored in a database. In the storing step 108, a photo of the patient's skin comprising the painful area and a pain intensity information may be stored together with the shape and the characteristic area size of the selected group. The photo of the skin of the patient may be taken before or after acquiring the pictures of the substrate. The pain intensity information may be received as a user input before or after acquiring the pictures of the substrate. An exemplary set of information to be stored in the database is depicted in FIG. 5. The set includes patient identifier 501, a symbol shape of the selected group 502, the photo 503 of the skin, the characteristic treatment area size 504 and a pain intensity information 505.

The method may additionally include a calculation step, in which a difference between the characteristic area size of the selected group and a predetermined characteristic area size is calculated. Thereby, a deviation from the predetermined characteristic area size may be indicated to the user. The predetermined characteristic area size may be a previously determined and stored characteristic area size, for example a characteristic area size determined at a previous treatment situation, e.g., a treatment situation one or more month ago. In a typical scenario, the characteristic area size decreases with ongoing treatments. Thus, the calculation step may serve to show the effect of recurrent treatments.

List of reference signs:

| | |
|---|---|
| 100 | method for monitoring a treatment area |
| 101 | marking step |
| 102 | acquisition step |
| 103 | detection step |
| 104 | determination step |
| 105 | grouping step |
| 106 | characterization step |
| 107 | selection step |
| 108 | storing step |
| 200 | substrate |
| 201 | treatment area |
| 202 | reference symbol |
| 203 | code |
| 300, 300′, 300″ | picture |
| 400, 400′, 400″ | group |
| 401 | closed contour area |
| 501 | patient identifier |
| 502 | symbol shape of the selected group |
| 503 | photo |
| 504 | characteristic area size |
| 505 | pain intensity information |

1. A method (100) for monitoring a treatment area of a patient, in particular a patient suffering from peripheral neuropathic pain, which patient is treated by cutaneous administration of a substance, in particular comprising capsaicin, in the treatment area, the method comprising the following method steps:
in a marking step (101), the treatment area is marked on a skin of the patient and/or on a substrate which is separate from the skin of the patient;
in an acquisition step (102), two or more pictures (300, 300′, 300″) of a scene are acquired, wherein the scene comprises the treatment area (201) marked in the marking step (101) and a predetermined reference symbol (202) having a predetermined reference area size;
in a detection step (103), closed contour areas (401) and the predetermined reference symbol (202) are detected in each of the pictures (300, 300′, 300″) acquired in the acquisition step (102);
in a determination step (104), a closed contour area size is determined for each closed contour area (401) detected in the detection step (103) from the respective detected closed contour area (401) and the predetermined reference symbol (202) detected in the same picture (300, 300′, 300″) as the closed contour area (401);
in a grouping step (105), the closed contour areas (401) detected in the detection step (103) are grouped according to their shape, thereby providing one or more groups (400, 400′, 400″) of closed contour areas (401);
in a characterization step (106), a characteristic area size (504) is determined for each group (400, 400′, 400″) of contour areas (401) identified in the grouping step (105) from the closed contour area sizes assigned to the closed contour areas (401) of each group (400, 400′, 400″);
in a selection step (107), a group of closed contour areas (400, 400′, 400″) is selected;
in storing step (108), the shape (502) and the characteristic area size (504) of the selected group (400, 400′, 400″) is stored in a database.

2. The method according to claim 1, wherein the substrate (200) is a foil or a tracing paper or a patch, in particular a patch comprising capsaicin.

3. The method according to claim 1, wherein, in the marking step (101), the treatment area (201) is marked as an outline or is marked as a colored area.

4. The method according to claim 1, wherein the predetermined reference symbol (202) is provided on the skin or on the substrate (200) or as a separate element.

5. The method according to claim 1, wherein, in the acquisition step (102), an orientation of an acquisition device acquiring the pictures is determined, in particular by a motion sensor.

6. The method according to claim 1, wherein, in the grouping step (105), the closed contour areas (401) detected in the detection step (103), in particular the shape and/or the closed contour area size of the closed contour areas (401) detected in the detection step, are compared to each other.

7. The method according to claim 1, wherein, in the grouping step (105), the closed contour areas (401) detected in the detection step are grouped according to their shape into groups (400, 400′, 400″) having similar shape.

8. The method according to claim 1, wherein, in the determination step (104), the predetermined reference symbol (202) is used as a calibration standard for determining the closed contour area size in the respective picture (300, 300′, 300″).

9. The method according to claim 1, wherein the characteristic area size (504) is a median area size or mean area size.

10. The method according to claim 1, wherein the selection step (107) comprises displaying the one or more groups (400, 400′, 400″) determined in the grouping step (105) to a user.

11. The method according to claim 1, wherein the acquisition step (102) and the detection step (103) and the determination step (104) and the grouping step (105) and the characterization step (106) are repeated before a group (400, 400′, 400″) of contour areas is selected in the selection step (107).

12. The method according to claim 1, wherein a photo (503) of the skin of the patient is taken, wherein the photo (503) is stored in the database in the storing step (108).

13. The method according to claim 1, wherein a pain intensity information (505) is received, wherein the pain intensity information (505) is stored in the database in the storing step (108).

14. The method according to claim 1, wherein calculation step, in which a difference between the characteristic area size (504) of the selected group and a predetermined characteristic area size is calculated.

15. A system for monitoring a treatment area of a patient, in particular a patient suffering from peripheral neuropathic pain, which patient is treated by cutaneous administration of a substance, in particular comprising capsaicin, in the treatment area, wherein the treatment area is marked on a skin of the patient and/or on a substrate which is separate from the skin of the patient,
the system comprising an acquisition device configured to acquire two or more pictures (300, 300′, 300″) of a scene, wherein the scene comprises the marked treatment area (201) and a predetermined reference symbol (202) having a predetermined reference area size;
the system comprising a processing device configured to carry out the following method steps:
in a detection step (103), closed contour areas (401) and the predetermined reference symbol (202) are detected in each of the pictures (300, 300′, 300″) acquired by the acquisition device;
in a determination step (104), a closed contour area size is determined for each closed contour area (401) detected in the detection step (103) from the respective detected closed contour area (401) and the predetermined reference symbol (202) detected in the same picture (300, 300′, 300″) as the closed contour area (401);
in a grouping step (105), the closed contour areas (401) detected in the detection step (103) are grouped according to their shape, thereby providing one or more groups (400, 400′, 400″) of closed contour areas (401);
in a characterization step (106), a characteristic area size (504) is determined for each group (400, 400′, 400″) of contour areas (401) identified in the grouping step (105) from the closed contour area sizes assigned to the closed contour areas (401) of each group (400, 400′, 400″);
in storing step (108), the shape (502) and the characteristic area size (504) of a selected group (400, 400′, 400″) is stored in a database.

16. The system according to claim 15, wherein, in the grouping step (105), the closed contour areas (401) detected in the detection step (103), in particular the shape and/or the closed contour area size of the closed contour areas (401) detected in the detection step, are compared to each other.

17. The system according to claim 15, wherein, in the grouping step (105), the closed contour areas (401) detected in the detection step are grouped according to their shape into groups (400, 400′, 400″) having similar shape.

18. The system according to claim 15, wherein, in the determination step (104), the predetermined reference symbol (202) is used as a calibration standard for determining the closed contour area size in the respective picture (300, 300′, 300″).

19. The system according to claim 15, wherein the characteristic area size (504) is a median area size or mean area size.

20. A method for treatment of a patient suffering from peripheral neuropathic pain, the method comprising:
cutaneous administration of capsaicin in a treatment area;
monitoring said treatment area of said patient wherein:
in an acquisition step (102), two or more pictures (300, 300′, 300″) of a scene are acquired, wherein the scene comprises the treatment area (201);
in a detection step (103), detecting closed contour areas (401);
determining a size and a location of a subsequent treatment area based on characterization of said closed contour areas (401);
cutaneous administration of capsaicin in said subsequent treatment area.

21. (canceled)
